(19) United States
(12) Patent Application Publication
(10) Pub. No.: US 2015/0119343 A1
(43) Pub. Date: Apr. 30, 2015

(54) Production of TSG-6 Protein

(71) Applicant: The Texas A & M University System, College Station, TX (US)

(72) Inventors: Darwin J. Prockop, Philadelphia, PA (US); Jun Watanabe, Temple, TX (US); Dong-Ki Kim, Temple, TX (US); RyangHwa Lee, Temple, TX (US); Hosoon Choi, Belton, TX (US)

(21) Appl. No.: 14/354,381

(22) PCT Filed: Nov. 1, 2012
(86) PCT No.: PCT/US12/62985

§ 371 Date: Apr. 25, 2014

Related U.S. Application Data
(60) Provisional application No. 61/555,681, filed on Nov. 4, 2011.

(51) Int. Cl. C07K 14/47; A61K 38/17

(52) U.S. Cl. CPC C07K 14/4718; A61K 38/1709
USPC 514/21.92; 435/69.1; 530/350

(57) ABSTRACT

A method of producing a protein or polypeptide, such as, for example, TSG-6 protein, or a biologically active fragment, derivative or analogue thereof, by introducing into mammalian cells a polynucleotide encoding the biologically active protein or polypeptide or biologically active fragment, derivative, or analogue thereof. The cells then are suspended in a protein-free medium that includes at least one agent that suppresses production of hyaluronic acid, hyaluronan, or a salt thereof by the cells. The cells are cultured for a time sufficient to express the biologically active protein or polypeptide or biologically active fragment, derivative or analogue thereof. The biologically active protein or polypeptide, or fragment, derivative, or analogue thereof then is recovered from the cells, such as, for example, by recovering the protein or polypeptide secreted by the cells from the cell culture medium.

This application claims priority based on provisional application Ser. No, 61/555,681. filed Nov. 4, 2011, the contents of which are incorporated by reference in their entirety.

This invention relates to the production of proteins or polypeptides such as, for example, TSG-6 protein, by mammalian cells. More particularly, this invention relates to the production of such proteins, and biologically active fragments, derivatives, and analogues thereof by introducing into mammalian cells a polynucleotide encoding a biologically active protein or polypeptide, or a biologically active fragment, derivative, or analogue thereof, and then culturing the cells by suspending the cells in a protein-free medium that includes at least one agent suppresses the production of hyaluronic acid or hyaluronan or a salt thereof by the cells. The cells are cultured for a period of time sufficient to express the biologically active protein or polypeptide, or a biologically active fragment, derivative, or analogue thereof. The biologically active protein or polypeptide, or a biologically active fragment, derivative, or analogue thereof then is recovered from the cultured cells.

Biologically active proteins and polypeptides, as well as fragments, derivatives, or analogues thereof, have a variety of therapeutic uses. Examples of such biologically active proteins and polypeptides include, but are not limited to, anti-inflammatory proteins, such as, for example, tumor necrosis factor stimulated gene 6 protein, or TSG-6, protein, anti-apoptotic proteins, such as, for example, stanniocalcin-1 and stanniocalcin-2, or STC-1 and STC-2, proteins, proteins that regulate cell growth and development, such as, for example, LIF protein; proteins that regulate hematopoiesis, such as, for example, IL-11, proteins that kill cancer cells or regulate immune response, such as, for example, TNFSF10 (also known as TRAIL), and IL-24; proteins that regulate homing of cells, such as, for example, CXCR4; proteins involved in cell adhesion and cell signaling, such as, for example, ITGA2 (also known as integrin α2); and proteins that enhance angiogenesis, such as, for example, IL-8.

Such biologically active proteins have a variety of therapeutic uses. For example, the anti-inflammatory protein, TSG-6, may be used to treat diseases and disorders of the eye, including macular degeneration, including age related macular degeneration (ARMD), and other maculopathies and retinal degeneration, corneal diseases and disorders, diseases and disorders of the anterior chamber of the eye, diseases and disorders of the iris, lens, and retina, eyelid diseases, lacrimal apparatus diseases, and glaucoma. TSG-6 also may be used to treat inflammation associated with myocardial infarction, stroke, Alzheimer's disease, atherosclerosis, and lung diseases.

Furthermore, TSG-6 may be used to treat inflammation associated with autoimmune diseases and immune pathologies, including rheumatoid arthritis, bacterial and/or viral infection, chronic inflammatory pathologies, vascular inflammatory pathologies, neurodegenerative disease, malignant pathologies involving INF-secreting tumors, and alcohol-induced hepatitis. (See, for example, U.S. Pat. Nos. 6,210,905 and 6.313,091).

The therapeutic proteins hereinabove described may be produced by a variety of techniques known to those skilled in the art, such as, for example, recombinant or genetic engineering techniques. For example, appropriate cells, such as, for example, mammalian cells, may be genetically engineered with a polynucleotide that encodes a biologically active protein or polypeptide, or a biologically active fragment, derivative, or analogue thereof. The cells then are cultured under conditions such that the cells express the biologically active protein or polypeptide, or a biologically active fragment, derivative, or analogue thereof.

Although biologically active proteins and polypeptides may be produced by recombinant techniques, some biologically active proteins and polypeptides, such as TSG-6, for example, are produced in limited quantities, and/or are difficult to recover from the cells which produce such proteins. Indeed, the ability to produce TSG-6 protein in sufficient amounts, to surmount the technical complexities, and to do so in a cost effective manner and efficiently has limited further study and development of TSG-6 protein, and of therapies employing TSG-6 protein.

The invention now will be described with respect to the drawings.

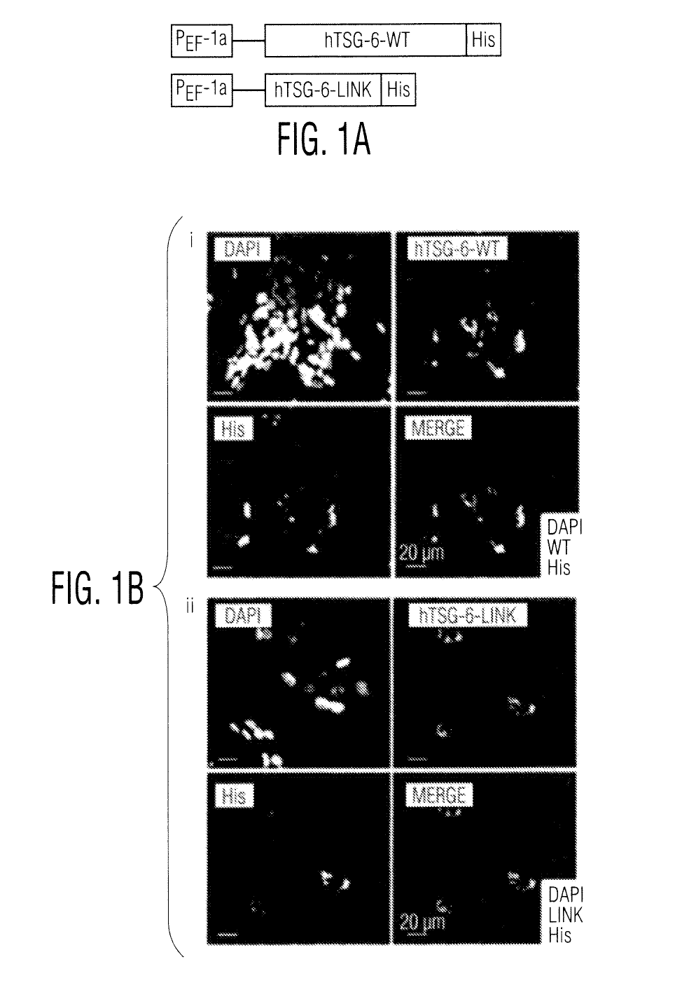

FIG. 1. Transiently transfected Chinese Hamster Ovary (CHO) cells express hTSG-6 wild-type and hTSG-6-LINK proteins. (A) Diagram of expression constructs encoding hTSG 6 wild-type protein or hTSG-6-LINK protein with a His-tag that are inserted into a pEF4/Myc-His expression vector. Each cDNA was fused to six histidine codons at its C-terminus under the control of a human elongation factor promoter (P_{EF}-1α). (B(i) and (ii)) After two days post-transfection, the transformed cells were labeled with anti-TSG and anti-His antibodies.

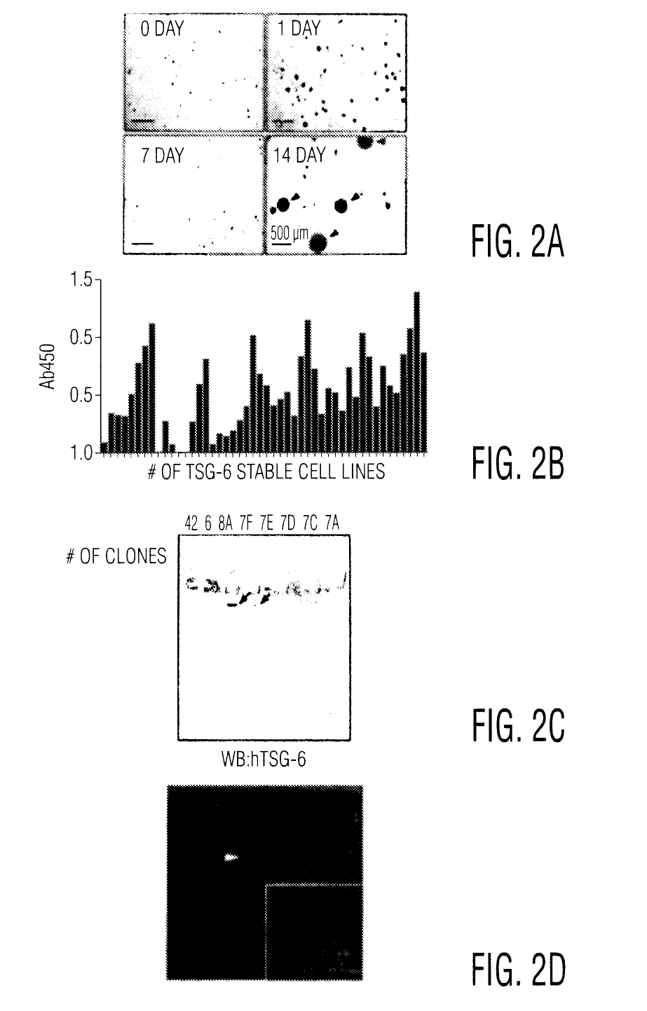

FIG. 2. Rapid establishment of hTSG-6/CHO stable cell lines using a methylcellulose-based formulation. (A) The cells were evaluated under a microscope at 0, 3. 7, and 14 days post-transfection. At 14 days post-transfection, the transformed clones that formed spheres were isolated under a microscope. (B) About 50 clones were analyzed for hTSG-6 protein secretion by an ELISA assay as a first screening step. Absorbance was measured at 450 nm. (C) Selected clones were analyzed for hTSG-6 protein secretion by a Western Blot assay. (D) The most productive clones were amplified further and as a final test, the expression of TSG-6 proteins within the clones was verified by immunocytochemistry with a fluorescent-labeled hTSG-6 antibody.

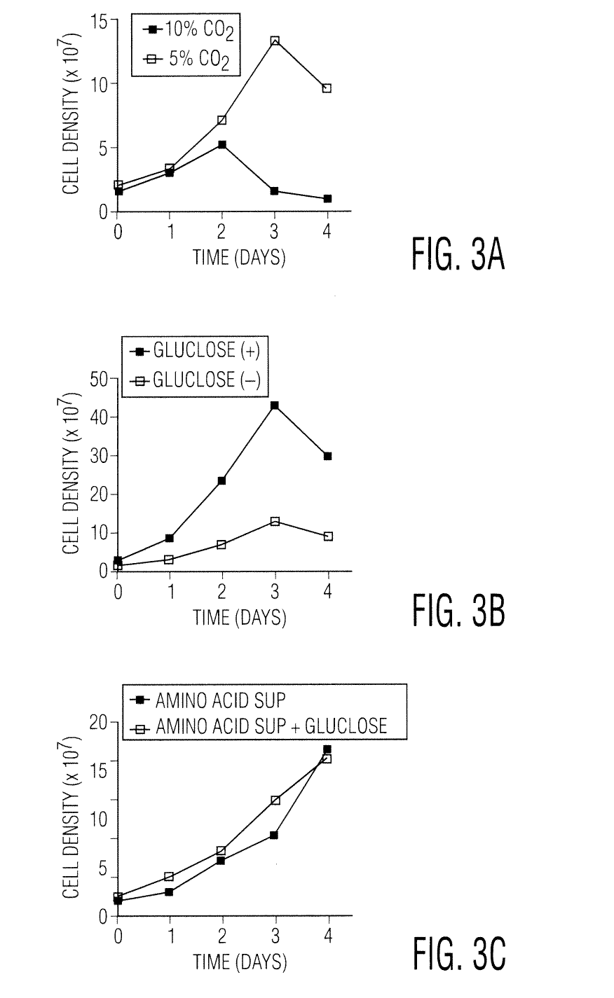

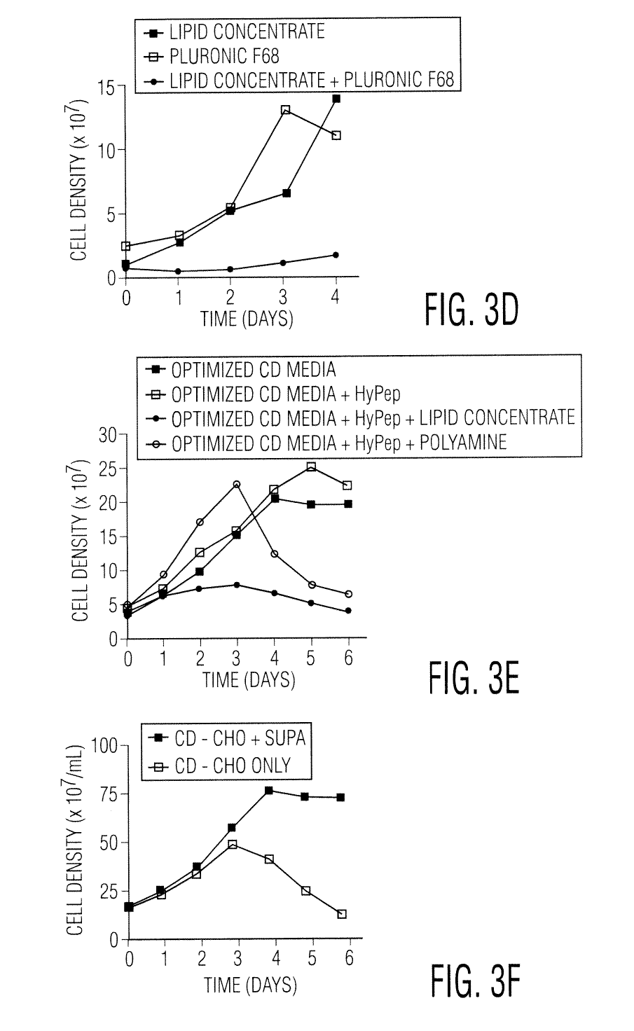

FIG. 3. Determination of optimal medium for spinner culture of rhTSG-6/CHO stable cell lines in chemical-defined protein free media supplemented with various factors. (A-E) (F) The optimal medium (Sup A) provided greater viability and survival than the standard CD-CHO medium.

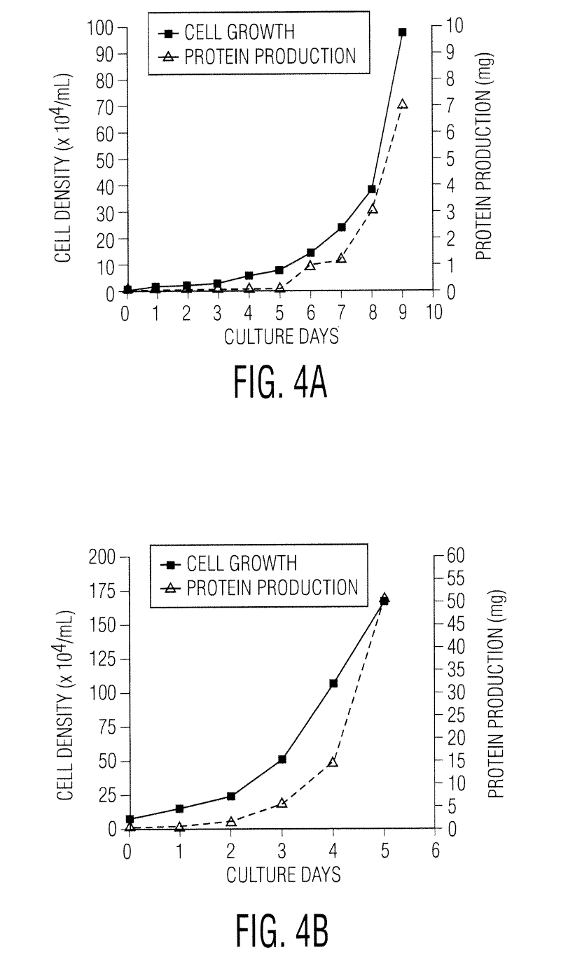

FIG. 4. Cell growth and TSG-6 yields in a bioreactor using the optimal medium (FIG. 3F). Cell seeding density was 5×10⁴ cells/ml. (A) 34° C. (B) 36° C.

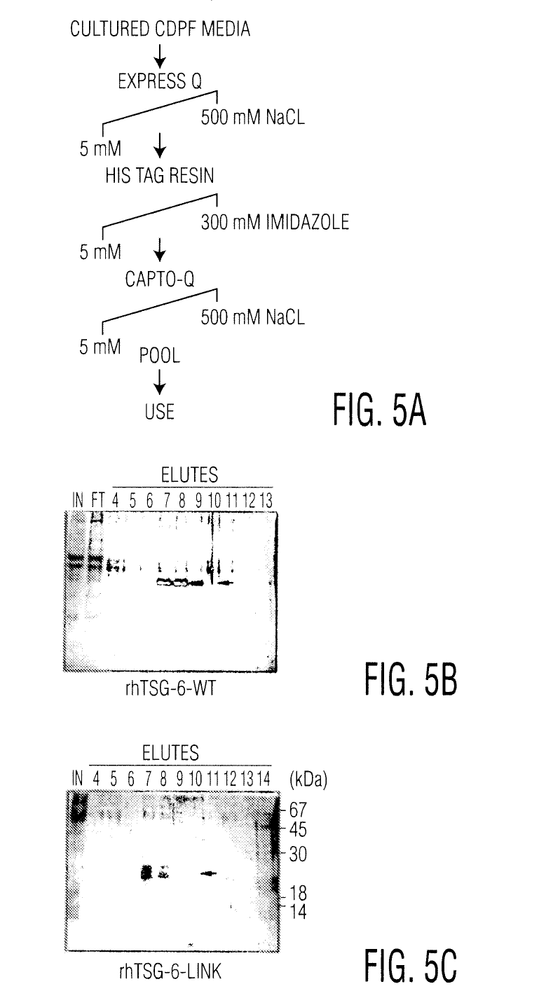

FIG. 5. Large scale purification of rhTSG-6 and its link module, hTSG-6-LINK- (A) protein purification steps of the cultured media of stable CHO cell lines. (B and C) SDS-PAGE profile of protein fractions. Multiple bands are detected with hTSG-6-LINK because of varying degrees of glycosylation.

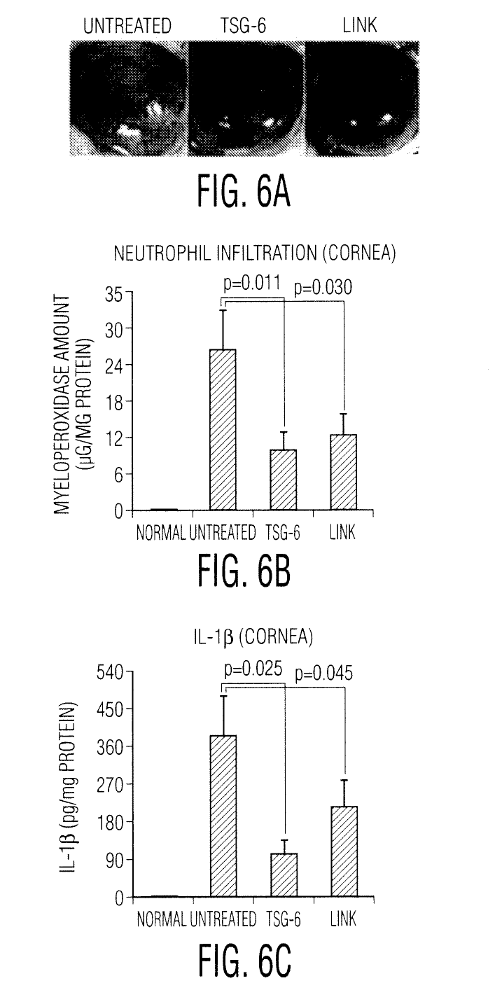

FIG. 6. rhTSG-6 and rhTSG-6-LINK reduced corneal opacity and inflammation in the cornea following injury. Corneas of rats were injured by 15 second exposure to ethanol followed by mechanical scraping of the epithelium and limbus, (Oh, et al., Proc. Nat. Acad. Sci., Vol. 107, No. 39, pgs. 16875-16880 (2010)). (A). Corneal opacity was reduced significantly in both rhTSG-6 and rhTSG-6-LINK-treated corneas. (B) For a quantitative measure of neutrophil infiltration, the concentration of myeloperoxidase (MPO) was assayed. Treatment with either rhTSG-6 or rhTSG-6-LINK reduced the levels of MPO in the cornea significantly. (C) The protein levels of the proinflammatory cytokine IL-1β were decreased significantly in the corneas treated with rhTSG-6 or rhTSG-6 LINK as assayed by ELISA.

It therefore is an object of the present invention to provide a more efficient method of producing recombinant biologically active proteins and polypeptides, and to produce such biologically active proteins and polypeptides in greater quantities.

In accordance with an aspect of the present invention, there is provided a method of producing a biologically active protein or polypeptide, or a biologically active fragment derivative, or analogue thereof. The method comprises introducing into cells, including, but not limited to, mammalian cells, a polynucleotide encoding a biologically active protein or polypeptide, or a biologically active fragment, derivative, or analogue thereof. The cells then are cultured by suspending the cells in a protein-free medium that includes at least one agent that suppresses production of hyaluronic acid or hyaluronan or a salt thereof by the cells. The cells are cultured for a time sufficient to express the biologically active protein or polypeptide, or a biologically active fragment, derivative, or analogue thereof. The expressed biologically active protein or polypeptide, or a biologically active fragment, derivative, or analogue thereof then is recovered from the cells.

In an alternative non-limiting embodiment, the medium in which the cells are cultured may contain protein, provided that the protein which is present does not interfere with the growth of the cultured cells, or interfere with optimal production of the biologically active protein or polypeptide, or biologically active fragment, derivative, or analogue thereof.

Thus, in accordance with another aspect of the present invention, there is provided a method of producing a biologically active protein or polypeptide, or a biologically active fragment, derivative, or analogue thereof. The method comprises introducing into cells, such as mammalian cells, a polynucleotide encoding a biologically active protein or polypeptide, or a biologically active fragment, derivative, or analogue thereof. The cells then are cultured by suspending the cells in a medium that includes at least one agent that suppresses production of hyaluronic acid or hyaluronan or a salt thereof by the cells. The cells are cultured for a time sufficient to express the biologically active protein or polypeptide, or a biologically active fragment, derivative, or analogue thereof. The expressed biologically active protein or polypeptide, or a biologically active fragment, derivative, or analogue thereof then is recovered from the cells.

In a non-limiting embodiment, the biologically active protein or polypeptide, or a biologically active fragment, derivative, or analogue thereof is a biologically active protein or polypeptide having a link domain or link module.

In another non-limiting embodiment, the biologically active protein or polypeptide is TSG-6 protein, or biologically active fragment, derivative, or analogue thereof. In another non-limiting embodiment, the biologically active protein or polypeptide includes the TSG-6 protein hyaluronan-binding link domain. The sequence of the “native” TSG-6 protein, having 277 amino acid residues, is given in the example hereinbelow. In one non-limiting embodiment, the link domain consists of amino acid residues 1 through 133. In another non-limiting embodiment, the link domain consists of amino acid residues 1 through 98, as described in Day, et al. Protein Expr. Purif, Vol. 1, pgs. 1-16 (Aug. 8, 1996).

The inflammation-associated protein TSG-6 cross-links hyaluronan via hyaluronan-induced TSG-6 oligomers. (Baranova, et al., J. Biol. Chem., Vol. 286, No. 29, pgs. 25675-25686 (Jul. 22, 2011; Epub, May 19, 2011). Tumor necrosis factor-stimulated gene 6 (TSG-6) is a hyaluronan-binding protein that plays important roles in inflammation and ovulation. TSG-6-mediated cross-linking of hyaluronan (HA) has been proposed as a functional mechanism (e.g., for regulating leukocyte adhesion) but direct evidence for cross-linking has been lacking. Full-length TSG-6 protein binds with pronounced positive cooperativity and it can cross-link HA at physiologically relevant concentrations. Cooperative binding of full-length TSG-6 arises from HA-induced protein oligomerization, and the TSG-6 oligomers act as cross-linkers. In contrast, the HA-binding domain of TSG-6 (i.e., the link module) alone binds without positive co-operativity and binds more weakly than the full-length protein. Both the link module and full-length TSG-6 protein condensed and rigidified HA films, and the degree of condensation scaled with the affinity between the TSG-6 constructs and HA. The condensation may be the result of protein-mediated HA cross-linking. TSG-6 is a potent HA cross-linking agent and may have important implications for the mechanistic understanding of the biological functions of TSG-6.

In another non-limiting embodiment, the biologically active protein or polypeptide or a biologically active fragment, derivative, or analogue thereof, such as TSG-6 protein or biologically active fragment, derivative, or analogue thereof, has a “His-tag” at the C-terminal thereof. The term “His-tag”, as used herein, means one or more histidine residues are bound to the C-terminal of the TSG-6 protein or biologically active fragment, derivative, or analogue thereof. In another non-limiting embodiment, the “His-tag” has six histidine residues at the C-terminal of the biologically active protein or polypeptide, such as TSG-6 protein or a biologically active fragment, derivative, or analogue thereof.

In a non-limiting embodiment, when the biologically active protein or polypeptide,or biologically active fragment, derivative, or analogue thereof, includes a “His-tag”, at the C-terminal thereof, the biologically active protein or polypeptide, or biologically active fragment, derivative, or analogue thereof, may include a cleavage site that provides for cleavage of the “His-tag” from the biologically active protein or polypeptide, or biologically active fragment, derivative, or analogue thereof, after the biologically active polypeptide, or biologically active fragment, derivative, or analogue thereof is produced.

The polynucleotide that encodes the biologically active polypeptide, or a biologically active fragment, derivative, or analogue thereof may be a DNA or RNA. Such polynucleotides include all nucleotides that are degenerate versions of each other and that encode the same amino acid sequence. The polynucleotide may include introns.

In general, the polynucleotide encoding the biologically active protein or polypeptide, or a biologically active fragment, derivative, or analogue thereof is part of a gene construct in which the polynucleotide encoding the biologically active protein or polypeptide, or a biologically active fragment, derivative, or analogue thereof is linked operatively to regulatory sequences to achieve expression of the polynucleotide in the mammalian cell. Such regulatory sequences including typically a promoter and a polyadenylation signal.

In a non-limiting embodiment, the gene construct is provided as an expression vector that includes the coding sequence for the biologically active protein or polypeptide which is linked operably to essential regulatory sequences such that when the vector is transfected into the cell, the coding sequence will be expressed by the mammalian cell. The coding sequence is linked operably to the regulatory elements necessary for expression of that sequence in the mammalian cells. The nucleotide sequence that encodes the biologically active protein or polypeptide may be cDNA, genomic DNA, synthesized DNA or a hybrid thereof, or an RNA molecule such as mRNA.

The gene construct includes the nucleotide sequence encoding the biologically active protein or polypeptide, which is linked operably to the regulatory elements and may remain present in the mammalian cell as a functioning cytoplasmic molecule, a functioning episomal molecule, or it may integrate into the mammalian cell's chromosomal DNA. Exogenous genetic material may be introduced into the cells where it remains as separate genetic material in the form of a plasmid. Alternatively, linear DNA which can integrate into the chromosome may be introduced into the mammalian cell. When introducing DNA into the mammalian cell, reagents which promote DNA integration into chromosomes may be added. DNA sequences which are useful to promote integration may also be included in the DNA molecule. Alternatively, RNA may be introduced into the mammalian cell.

The regulatory elements for gene expression include: a promoter, an initiation codon, a stop codon, and a polyadenylation signal. It is preferred that these elements be operable in the mammalian cells of the present invention. Moreover, it is preferred that these elements be linked operably to the nucleotide sequence that encodes the protein or polypeptide such that the nucleotide sequence can be expressed in the cells and thus the protein can be produced. Initiation codons and stop codons are considered generally to be part of a nucleotide sequence that encodes the protein or polypeptide; however, it is preferred that these elements are functional in the mammalian cells. Similarly, promoters and polyadenylation signals used must be functional within the cells of the present invention. Examples of promoters useful to practice the present invention include, but are not limited to, promoters that are active in many cells such as the cytomegalovirus promoter, SV40 promoters, and retroviral promoters. In some non-limiting embodiments, promoters are used which express genes in the mammalian cells constitutively with or without enhancer sequences. Enhancer sequences are provided in such embodiments when appropriate or desirable.

In a non-limiting embodiment, the polynucleotide encoding the biologically active protein or polypeptide, or biologically active fragment, derivative, or analogue thereof is contained in a pEF4/Myc-His expression vector, (Invitrogen). Such vectors include a human elongation factor la-subunit (hEF-1α) promoter which controls expression of the polynucleotide encoding the biologically active protein or polypeptide or a biologically active fragment, derivative, or analogue thereof, a multiple cloning site, a C-terminal tag encoding a polyhistidne (6 histidne residues) metal binding polypeptide, a Zeocin resistance gene flanked by an SV40 origin of replication and an SV40 poly A signal, and an ampicillin resistance gene.

The mammalian cells of the present invention can be transfected using well known techniques readily available to those having ordinary skill in the art. Exogenous genes may be introduced into the cells using standard methods where the cell expresses the protein encoded by the gene. In some embodiments, mammalian cells are transfected by calcium phosphate precipitation transfection, DEAE dextran transfection, electroporation, microinjection, liposome-mediated transfer, chemical-mediated transfer, ligand mediated transfer or recombinant viral vector transfer.

In some non-limiting embodiments, recombinant adenovirus vectors are used to introduce DNA with desired sequences into the mammalian cell. In some non-limiting embodiments, recombinant retrovirus vectors are used to introduce DNA with desired sequences into the mammalian cells. In other embodiments, standard CaPO₄, DEAF, dextran or lipid carrier mediated transfection techniques are employed to incorporate desired DNA into dividing mammalian cells. In some non-limiting embodiments. DNA is introduced directly into the mammalian cells by microinjection. Similarly, well-known electroporation or particle bombardment techniques can be used to introduce foreign DNA into the cells. A second gene may be co-transfected with, or linked to the polynucleotide encoding the biologically active protein or polypeptide. The second gene frequently is a selectable marker, such as a selectable antibiotic-resistance gene. Standard antibiotic resistance selection techniques can be used to identify and select transfected biologically active protein or polypeptide cells. Transfected cells are selected by growing the cells in an antibiotic that will kill cells that do not take up the selectable gene. In most cases where the two genes co-transfected and unlinked, the cells that survive the antibiotic treatment contain and express both genes.

In another non-limiting embodiment, the polynucleotide encoding the biologically active protein or polypeptide is contained in an expression cassette, and is linked operably to a suitable promoter.

The expression cassette containing the polynucleotide encoding the biologically active protein or polypeptide should be incorporated into the genetic vector suitable for delivering the transgene to the mammalian cell. Depending on the desired end application, any such vector can be so employed to modify the cells genetically (e.g., plasmids, naked DNA, viruses such as adenovirus, adeno-associated virus, herpesvirus, lentivirus. papillomavirus, retroviruses, etc.). Any method of constructing the desired expression cassette within such vectors can be employed, many of which are well known in the art, such as by direct cloning, homologous recombination, etc. The desired vector will determine largely the method used to introduce the vector into the cells, which are generally known in the art. Suitable techniques include protoplast fusion, calcium-phosphate precipitation, gene gun, electroporation, and infection with viral vectors.

Mammalian cells which may be employed include any mammalian cell into which may be introduced a polynucleotide encoding a biologically active protein or polypeptide, or a biologically active fragment, derivative, or analogue thereof. In a non-limiting embodiment, the mammalian cells are Chinese hamster ovary, or CHO, cells.

Alternatively, the polynucleotide encoding a biologically active protein or polypeptide, or biologically active fragment, derivative, or analogue thereof, may be introduced into other eukaryotic cells, such as yeast cells, or prokaryotic cells, such as E. coli cells, for example.

The cells which include the polynucleotide encoding the biologically active protein or polypeptide are suspended in an appropriate protein-free medium that includes at least one agent that suppresses production of hyaluronic acid or hyaluronan or a salt thereof by the cells.

In a non-limiting embodiment, the at least one agent that suppresses production of hyaluronic acid or hyaluronan or a salt thereof by the mammalian cells is 4-methylumbelliferone, also known as MU or 7-hydroxy-4 methyl-2H-1-benzopyran-2-one. Although the scope of the present invention is not to be limited to any theoretical reasoning, certain biologically active proteins or polypeptides, such as TSG-6 and fragments, derivatives, or analogues thereof, bind to hyaluronic acid or hyaluronan or a salt thereof, produced by the cells, and thus are secreted by the cell in reduced quantities. By suppressing the production of hyaluronic acid or hyaluronan or a salt thereof, the 4-methylumbelliferone may enable the cells to produce and secrete increased amounts of the biologically active protein or polypeptide, such as TSG-6 protein or a biologically active fragment, derivative, or analogue thereof, or may allow higher synthesis, or better recovery and separation of the biologically active protein or polypeptide from the cells.

In other non-limiting embodiments, the at least one agent that suppresses production of hyaluronic acid or hyoluronan or a salt thereof by the cells is an antisense polynucleotide or small interfering RNA (siRNA) that blocks hyaluronan synthesis, or an antibody that binds to hyaluronan.

In another non-limiting embodiment, the protein-free medium is free of plasma.

In a further non-limiting embodiment, the protein-free medium includes chemically defined CHO medium, hypoxanthine/thymine, or HT, L-glutamine, glucose (such as, for example, D-(+)-glucose), 4-methylumbelliferone, non-essential amino acids, MEM (Minimal Essential Medium) vitamin solution, penicillin, and streptomycin.

The cells are cultured under conditions and for a time sufficient to express the biologically active protein or a biologically active fragment, derivative, or analogue thereof in a desired amount. In a non-limiting embodiment, the cells are cultured at a temperature of about 36° C. In another non-limiting embodiment, the cells are cultured for a total period of time of from about 2 days to about 14 days. In yet another non-limiting embodiment, the cells are cultured for a total period of time of from about 4 days to about 10 days.

In a non-limiting embodiment, the cells are transfected with a pEF4/Myc-His vector which includes the polynucleotide encoding a biologically active protein or polypeptide or fragment, derivative, or analogue thereof. The transfected cells then are plated onto a medium containing fetal bovine serum (FBS) and Iscove's Modified Dulbecco's Medium. (IMDM), and Zeocin. The cells are cultured until they reach a cell density of about 90%.

The cells then are cultured in a spinner bottle, whereby the cells are suspended in a protein-free medium such as hereinabove described, and which includes at least one agent, e.g., 4-methylumbelliferone, that suppresses production of hyaluronic acid by the cells. The cells are cultured at a temperature of 36° C. until they reach an appropriate cell density, such as, for example, about 3 to 60×10⁴ cells/ml. In a non-limiting embodiment, such period of time is about 4 days.

The cells then are suspended in the protein-free medium, such as hereinabove described, in a bioreactor. A pH control reagent, such as NaOH, may be added to the medium to maintain the pH of the medium at about 7.4. The cells are cultured in the bioreactor until they reach an appropriate cell density, such as, for example, about 175-220×10⁴ cells/ml. In a non-limiting embodiment, such period of time is at least about 5 days.

The cultured medium then is collected from the bioreactor, and the biologically active protein or polypeptide, such as TSG-6 protein or a biologically active fragment, derivative, or analogue thereof, such as a TSG-6 protein having a His-tag of 6 histidine residues at the C-terminal thereof, is recovered from the cultured medium. Such recovery may be effected by any of a variety of means known to those skilled in the art. Such methods include, but are not limited to, ion exchange gradient columns used in combination with an appropriate buffer, and the like. When the protein or polypeptide includes a His-tag at the C-terminal thereof, a column containing a nickel chelate His-tag resin also may be employed as part of the protein recovery process.

The biologically active proteins or polypeptides, or a biologically active fragments, derivatives, or analogues thereof, that are produced and recovered in accordance with the present invention, may be employed in their respective therapeutic uses. For example, in a non-limiting embodiment, TSG-6 protein, or TSG-6 protein or biologically active fragment, derivative, or analogue thereof, including TSG-6 protein or fragment, derivative, or analogue thereof that includes a “His-tag” at the C-terminal thereof, may be used in any of the therapeutic applications hereinabove described for TSG-6 protein, including the treatment of diseases or disorders of the eye.

Applicants have discovered that, when TSG-6 protein, or a biologically active fragment, derivative, or analogue thereof, includes a “His-tag” at the C-terminal thereof, such TSG-6 protein or a fragment, derivative, or analogue thereof having a “His-tag” at the C-terminal thereof, has the same biological activity as a “native” TSG-6 protein or biologically active fragment, derivative, or analogue thereof.

For example, the TSG-6 protein or biologically active fragment, derivative, or analogue thereof, including TSG-6 protein having a His-tag at the C-terminal thereof, may be used to treat various ocular diseases or conditions, including the following: maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, Lyme Disease, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease, Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (PONS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Best's disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

An anterior ocular condition is a disease, ailment or condition which affects or which involves an anterior (i.e. front of the eye) ocular region or site, such as a periocular muscle, an eyelid or an eyeball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract: conjunctival diseases; conjunctivitis, including, but not limited to, atopic keratoconjunctivitis; corneal injuries, including, but not limited to, injury to the corneal stromal areas; corneal diseases; corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

A posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site. Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors: retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy. non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

Other diseases or disorders of the eye which may be treated with the TSG-6 protein or biologically active fragment, derivative, or analogue thereof, including a TSG-6 protein or biologically active fragment, derivative, or analogue thereof having a His-tag of 6 amino acid residues at the C-terminal thereof, include, but are not limited to, ocular cicatricial pemphigoid (OCP), and cataracts.

In a non-limiting embodiment, when inflammation of and/or injury to and/or disease or disorder of the eye is associated with an infection, e.g., a bacterial, viral, or fungal infection, the TSG-6 protein or biologically active fragment, derivative, or analogue thereof may be administered in combination with at least one anti-infective agent.

In general, at least one anti-infective agent which is administered in combination with the TSG-6 protein or biologically active fragment, derivative, or analogue thereof depends upon the type of infection, e.g., bacterial, viral, or fungal, to the eye the type or species of bacterium, virus, or fungus associated with the infection, and the extent and severity of the infection, and the age, weight, and sex of the patient.

In a non-limiting embodiment, when the infection of the eye is associated with one or more bacteria, the at least one anti-infective agent which is administered in combination with the TSG-6 protein or biologically active fragment, derivative, or analogue thereof is at least one anti-bacterial agent. Anti-bacterial agents which may be administered include, but are not limited to, quinolone antibiotics, such as, for example, ciprofloxacin, levofloxacin (Cravit), moxifloxacin (Vigamox), gatifloxacin (Zy-mar), cephalosporin, aminoglycoside antibiotics (e.g., gentamycin), and combinations thereof.

In another non-limiting embodiment, when the infection of the eye is associated with one or more viruses, the anti-infective agent which is administered in combination with the TSG-6 protein or biologically active fragment, derivative, or analogue thereof is at least one anti-viral agent. Anti-viral agents which may be employed include those which are known to those skilled in the art.

In another non-limiting embodiment, when the infection of the eye is associated with one or more fungi, the anti-infective agent which is administered in combination with the TSG-6 protein or biologically active fragment, derivative, or analogue thereof is at least one anti-fungal agent. Anti-fungal agents which may be employed include, but are not limited to, natamycin, amphotericin B, and azoles, including fluconazole and itraconzole.

In yet another non-limiting embodiment, when the infection of the eye is associated with more than one of bacteria, viruses, and fungi, more than one of anti-bacterial, anti-viral, and anti-fungal agents are administered in combination with the TSG-6 protein or biologically active fragment, derivative, or analogue thereof.

In a non-limiting embodiment, the TSG-6 protein or biologically active fragment, derivative, or analogue thereof may be administered to a patient in combination with other therapeutic agents employed in treating macular degeneration. Such therapeutic agents include, but are not limited to, angiogenesis inhibitors, and anti-vascular endothelial growth factor A (VEGF-A) antibodies (e.g., Avastin, Lucentis), agents or drugs which bind angiogenic agents, such as VEGF trap agents, tyrosine kinase inhibitors, which are anti-angiogenic, angiogenic protein receptor antagonists, and antibodies and antibody fragments which recognize heat shock proteins, including, but not limited to antibodies and antibody fragments which recognize the small heat shock protein HSPB4, HSP90, HSP70, HSP65, or HSP27, and heat shock protein antagonists, including, but not limited to, antagonists to HSPB4, HSP90, HSP70, HSP65, and HSP27.

Administration of the TSG-6 protein or biologically active fragment or derivative or analogue thereof typically is parenteral, by intravenous, subcutaneous, intramuscular, or intraperitoneal injection, or by infusion or by any other acceptable systemic method. In a non-limiting embodiment, the TSG-6 protein or biologically active fragment, derivative, or analogue thereof is provided to a mammal by intraocular administration. In a non-limiting embodiment, administration is by intravenous infusion, typically over a time course of about 1 to 5 hours. In addition, there are a variety of oral delivery methods for the administration of the TSG-6 protein or biologically active fragment, derivate or analogue thereof.

Alternatively, in a non-limiting embodiment, the TSG-6 protein or biologically active fragment, derivative, or analogue thereof may be administered to the eye topically, such as, for example, in the form of eye drops. In a further non-limiting embodiment,eye drops which include the TSG-6 protein or an analogue or fragment or derivative thereof, are administered to the cornea in order to treat or prevent a disease or disorder of the cornea.

In another non-limiting embodiment, the TSG-6 protein or biologically active fragment, derivative, or analogue thereof may be administered systemically, such as by intravenous administration, or intraocularly, such as by intracameral administration, to the anterior chamber of the eye.

Often, treatment dosages are titrated upward from a low level to optimize safety and efficacy. Generally, daily dosages will fall within a range of about 0.01 to 20 mg protein per kilogram of body weight. Typically, the dosage range will be from about 0.1 to a mg protein per kilogram of body weight.

Various modifications or derivatives of the TSG-6 protein or biologically active fragment, derivative, or analogue thereof, such as addition of polyethylene glycol chains (PEGylation), may be made to influence their pharmacokinetic and/or pharmacodynamic properties.

To administer the TSG-6 protein or biologically active fragment, derivative, or analogue thereof, by other than parenteral administration, the protein may be coated or co-administered with a material to prevent its inactivation. For example,the TSG-6 protein or biologically active fragment, derivative or analogue thereof, may be administered in an incomplete adjuvant, co-administered with enzyme inhibitors or administered in liposomes. Enzyme inhibitors include pancreatic trypsin inhibitor, disopropylfluorophosphate (DEP) and trasylol. Liposomes include water-in-oil-in-water, CGF emulsions, as well as conventional liposomes (Strejan, et al., (1984) J. Neuroimmunol. 7:27).

An “effective amount” of the TSG-6 protein or biologically active fragment, derivative, or analogue thereof, is an amount that will ameliorate one or more of the well known parameters that characterize medical conditions such as inflammation associated with the cornea, as well as the other diseases and disorders of the eye hereinabove described. An effective amount, in the context of inflammatory diseases of the cornea, as well as the other diseases or disorders hereinabove described, is the amount of protein or fragment, derivative, or analogue thereof that is sufficient to accomplish one or more of the following: decrease the severity of symptoms; decrease the duration of disease exacerbations; increase the frequency and duration of disease remission/symptom-free periods; prevent fixed impairment and disability; and/or prevent/attenuate chronic progression of the disease.

Although the compositions of this invention can be administered in simple solution, they are more typically used in combination with other materials such as carriers, preferably pharmaceutical carriers. Useful pharmaceutical carriers can be any compatible, non-toxic substance suitable for delivering the compositions of the invention to a patient. Sterile water, alcohol, fats, waxes, and inert solids may be included in a carrier. Pharmaceutically acceptable adjuvants (buffering agents, dispersing agents) may also be incorporated into the pharmaceutical composition. Generally, compositions useful for parenteral administration of such drugs are well known; e.g., Remington's Pharmaceutical Science, 17th Ed. (Mack Publishing Company, Easton, Pa., 1990). Alternatively, compositions of the invention may be introduced into a patient's body by implantable drug delivery systems [Urquhart et al., Ann. Rev. Pharmacol. Toxicol. 24:199 (1984).

Therapeutic formulations may be administered in many conventional dosage formulations. Formulations typically comprise at least one active ingredient, together with one or more pharmaceutically acceptable carriers.

The formulations conveniently may be presented in unit dosage form and may be prepared by any methods well known in the art of pharmacy. See, e.g., Gilman et al. (eds.) (1990), The Pharmacological Bases of Therapeutics, 8th Ed Pergamon Press; and Remington's Pharmaceutical Sciences, supra, Easton, Pa.; Avis, et al. (eds.) (1993) Pharmaceutical Dosage Forms: Parenteral Medications Dekker, N.Y.; Lieberman et al. (eds.) (1990), Pharmaceutical Dosage Forms: Tablets, Dekker, N.Y.; and Lieberman et al. (eds.) (1990), Pharmaceutical Dosage Forms: Disperse Systems, Dekker, N.Y.

Therapeutic compositions and formulations thereof of the invention can be used, for example, for reducing inflammation due to seasonal or bacterial conjunctivitis, for reducing post-surgical pain and inflammation, to prevent or treat fungal or bacterial infections of the eye, to treat herpes ophthalmicus, to reduce intraocular pressure, or to treat endophthalmitis.

More particularly, in one non-limiting embodiment, the present invention provides a method for treating an ophthalmic disorder in a mammal (e.g., including human and non-human primates), the method comprising administering to the eye of the mammal a therapeutically effect amount of a formulation of the present invention comprising a lipid phase, an aqueous phase and a TSG-6 protein or biologically active fragment, derivative, or analogue thereof as hereinabove described, wherein the protein or biologically active fragment, derivative, or analogue thereof, is useful for treating the ophthalmic disorder. In one embodiment, the ophthalmic disorder is post-operative pain. In another embodiment, the ophthalmic disorder is ocular inflammation resulting from, e.g., iritis, conjunctivitis, seasonal allergic conjunctivitis, acute and chronic endophthalmitis, anterior uveitis, uveitis associated with systemic diseases, posterior segment uveitis, chorioretinitis, pars planitis, masquerade syndromes including ocular lymphoma, pemphigoid. scleritis, keratitis, severe ocular allergy, corneal abrasion and blood-aqueous barrier disruption. In yet another embodiment, the ophthalmic disorder is post-operative ocular inflammation resulting from, for example, photorefractive keratectomy, cataract removal surgery, intraocular lens implantation and radial keratotomy.

In employing the liposome formulations of the present invention, in a non-limiting embodiment, administration is ocularly, which term is used to mean delivery of therapeutic agents through the surface of the eye, including the sclera, the cornea, the conjunctiva and the limbus, or into the anterior chamber of the eye. Ocular delivery can be accomplished by numerous means, for example, by topical application of formulation such as an eye drop, by injection, or by means of an electrotransport drug delivery system.

In another non-limiting embodiment, the TSG-6 protein or biologically active fragment, derivative, or analogue thereof employed for creating a disease or disorder of the eye may be contained in a nanoparticle. Such nanoparticles may be formed by methods known to those skilled in the art.

Such nanoparticles may be administered ocularly, i.e., through the surface of the eye, including the sclera, cornea, conjunctiva, and the limbus, or into the anterior chamber of the eye. Such ocular administration may be accomplished by any of a variety means, including, in a non-limiting embodiment, by topical application of a formulation such as an eye drop, by injection, or by means of an electotransport drug delivery system.

The invention now will be described with respect to the following example; it is to be understood, however, that the scope of the present invention is not intended to be limited thereby.

Material and Methods

hMSC's Culture

Frozen vials of human mesenchymal stem cells (hMSCs) from bone marrow were obtained from the Center for the Preparation and Distribution of Adult Stem Cells (formerly http://www.com.tulane.edu/gene_therapy/distribute.shtml; currently http://medicine.tamhsc.edu/irm/msc-distribution.html) that supplies standardized preparations of MSCs enriched for early progenitor cells to over 300 laboratories under the auspices of an NIH/NCRR grant (P40 RR 17447-06). A frozen vial of 10⁶ passage 1 cells was thawed, and plated at 200 to 500 cells/cm² in 150 mm plates with 30 mL of complete culture medium (CCM) that consisted of α-minimal essential medium (α-MEM; Invitrogen, Carlsbad, Calif.), 17% fetal bovine serum (FBS; lot-selected for rapid growth of MSCs; Atlanta Biologicals, Inc., Norcross, Ga.), 100 units/mL penicillin, 100 streptomycin, and 2 mM L-glutamine (Invitrogen). The cultures were incubated for approximately 5 days until they were 70% confluent with replacement of medium every 2 days. The cultures were washed with PBS and the cells harvested by incubation for 5 to 10 min. at 37° C. with 0.25% trypsin and 1 mM EDTA.

In order to up-regulate expression of TSG-6, the MSCs were expanded to about 70% confluency and then incubated at 37° C. for 24 hours in α-MEM containing 20 ng/mL TNF-α, 2% FBS, 100 units/mL penicillin, 100 μg/mL streptomycin, and 2 mM L-glutamine (Lee et al., Cell Stem Cell, Vol. 5, pgs. 54-63 (2009)).

Plasmid Construction

Total RNA was isolated from TNF-α stimulated hMSC cells (3×10⁴ cells/cm) and one microgram of total RNA was used to produce the first strand cDNA pool by RT-PCR (Superscript II/oligo dT₁₂₋₁₈, Invitrogen). cDNA encoding hTSG-6 (GenBank accession number: NM_{—}007115) was amplified by PCR. Primer sequences for the hTSG-6 genes that were cloned were 5′-CGGGGTACCATGATCATCTTAATTTACTT-3′ (sense for hTSG-6-WT and -LINK), 5′-GGTGATCAGTGGCTAAATCTTCCA-3′ (anti-sense for hTSG-6-WT), and 5′-GGAGTACTCTTTGCGTGTGGGTTGTAGCA-3′ (antisense for hTSG-6-LINK). The TSG-6 protein has the following amino acid sequence shown below. The TSG-6-LINK protein, or TSG-6 link module domain, consists of amino acid residues 1 through 133 hereinbelow:

MIILIYLFLL LWEDTQGWGF KDGIFHNSIW LERAAGVYHR
EARSGKYKLT YAEAKAVCEF EGGHLATYKQ LEAARKIGFH
VCAAGWMAKG RVGYPIVKPG PNCGFGKTGI IDYGIRLNRS
ERWDAYCYNP HAKECGGVFT DPKQIFKSPG FPNEYEDNQI
CYWHIRLKYG QRIHLSFLDF DLEDDPGCLA DYVEIYDSYD
DVHGFVGRYC GDELPDDIIS TGNVMTLKFL SDASVTAGGF
QIKYVAMDPV SKSSQGKNTS TTSTGNKNFL AGRFSHL

The PCR products were subcloned into the BamIII and EcoRI sites in the multiple cloning site of a pEF4/Myc/His plasmid (Invitrogen, Carlsbad, Calif.). Thus, the resulting pEF4/Myc-His plasmid vectors include DNA encoding hTSG-6 wild-type(WT) or hTSG-6-LINK protein under the control of the P_{EF}-1α promoter, each of which has a DNA sequence encoding a His-tag of 6 histidine residues at the 3′ end. (FIG. 1A).

Establishment of rh TSG-6-WT and -LINK CHO Stable Cell Lines

Chinese Hamster Ovary (CHO)-S cells were plated at 1×10⁵ cells in a 100 mm culture dish in 10 ml. IMDM (Iscove's Modified Dulbecco's Medium) containing 5% FBS, 50 units/ml of penicillin, and 50 μg/ml of streptomycin. After incubation for 2 days, cells were transfected with 30 μg of the constructed expression vector for rhTSG-6-WT or rhTSG-6-LINK using 20 μl of Lipofectamine 2000™ (invitrogen) in serum-reduced Opti media (Invitrogen). Four hours later, the medium was replaced with 10 ml of 5% FBS/IMDM and further incubated for one day. In order to determine whether the cells were expressing TSG-6 or TSG-6-LINK protein, the cells were labeled with DAPI and fluorescent antibodies which bind to TSG-6 or histidine. As shown in FIGS. 1B(i) and (ii), it was determined that the transfected cells expressed TSG-6 or TSG-6-LINK protein. The next day, the transfected cells were lifted and reseeded in a 100 mm culture dish in 9 mL ClonaCell-TCS medium (StemCell technologies) containing 500 μg/ml of Zeocin to select transformed clones. The cells were cultured further for 14 days, a time sufficient for the clones to form spheres in the methylcellulose-based semi-solid selection media.

The clones were examined under a microscope at 0, 3, 7, and 14 days post-transfection. After 14 days post-transfection, the transformed clones that form spheres were isolated under a microscope using a pipette. (FIG. 2A). About 50 clones then were tested and analyzed for TSG-6 protein secretion by ELISA, in which absorbance was measured at 450 nm. (FIG. 2B), Selected clones, i.e., clones 42, 6, 8A, 7F, 7E, 7D, 7C, and 7A, then were analyzed for TSG-6 protein secretion by Western Blot. (FIG. 2C). The most productive clones then were amplified further by plating on 15 cm diameter dishes in CCM and culturing for 2 days, and as a final test, the expression of TSG-6 protein within the clones was verified by immunocytochemistry with a fluorescent-labeled anti hTSG-6 antibody. (FIG. 2D).

The optimal medium for culturing rhTSG-6/CHO cell lines was determined by incubating the cell lines in a spinner bottle by seeding the cells in a chemically defined protein free medium (CDPF) that included 1 liter of CHO medium (CD-CHO, cat. #10743-011; Invitrogen), either alone (FIG. 3F), or in combination with 5% or 10% CO₂ (FIG. 3A); D-(+)-glucose or D-(−)-glucose (FIG. 3B); 10 ml non-essential amino acids or non-essential amino acids in combination with glucose (FIG. 3C); lipid concentrate. Pluronic F68, or lipid concentrate and Pluronic F68 (FIG. 3D); 10 ml hypoxanthine/thymidine medium (HT 100×, or HyPep cat. #11067-030, Invitrogen), or Hy Pep and lipid concentrate, or Hy Pep and polyamine (FIG. 3E). As indicated in FIG. 3F, the cells also were cultured in a medium referred to as CD-CHO+ SupA, which is a chemically defined protein free medium (CDPF) that was prepared with 1 liter CHO medium (CD-CHO cat. #10743-011; Invitrogen), 10 mL hypoxathine/thymidine medium (HT 100×. cat. #11067-030; lnvitrogen), 40 mL L-glutamine (final concentration 8 mM; L-Glutamine 200 mM; cat, #G6152-100G; Sigma); 2 grams D-(+)-glucose (cat. # G6152-100G; Sigma). 10 mL non-essential amino acids (cat. 11140-050; Invitrogen), 10 mL MEM vitamin solution (cat. #11120-052; Invitrogen), 5 mL penicillin/streptomycin (10,000 units Penicillin and 10,000 μg Streptomycin; cat. #15140163; Invitrogen) and 4-methylumbelliferone added to a 50 μM concentration (Wako Pure Chemicals; Osaka, Japan).

The cells were cultured in the various media hereinabove described for a period of time of from 4 days to 6 days, after which cell densities were measured. As shown in FIG. 3F, the cells that were cultured in the CD-CHO+ Sup A medium had greater viability and survival than cells cultured in the other media shown in FIGS. 3A through 3E.

The most productive clones were expanded in a spinner bottle by seeding about 3×10⁴ cells/mL in 500 mL in 5 liters of CDPF medium (i.e., CD-CHO+ Sup A).

In order to determine the optimum temperature for culturing the cells in a bioreactor, the cells then were seeded at 5×10⁴ cells/ml in 5 liters of the CDPF medium (CD-CHO+ Sup A) and incubated at a temperature of 34° C. or 36° C. for up to 9 days. (FIGS. 4A and 4B) in a bioreactor (Pilot Plant System; W350040-A Wheaton Science Products; 10 liter capacity). As shown in FIG. 4B, after 5 days, the cells that were incubated at 36° C. had a cell density of about 175×10⁴ cells ml, and produced about 50 mg of protein.

Purification of Secreted Proteins

The more productive clones were suspended at 5×10⁴ cells/ml in 5 liters of the CDPF medium (CD-CHO+ Sup A) hereinabove described in the bioreactor hereinabove described, for up to 8 days. The medium was clarified by centrifugation at 10,000 rpm for 10 min. Proteins were purified from the culture medium by sequential chromatography on an ion exchange column (300 mL resin bed; Express Ion Exchanger Q; Whatman/GE Healthcare, UK) eluted with 5 to 500 mM NaCl, and then a histidine binding nickel chelate column (25 mL resin bed; Ni-NTA agarose; Qiagen) eluted with 300 mM imidazole. The peak fractions were diluted 10-fold with 50 mM Tris-HCl (pH 7.4) and chromatographed on a second ion exchange column (10 mL resin bed; Capto Q; Pharmacia Biotech) eluted with 5 to 500 mM NaCl. (FIG. 5A). About 15 fractions were collected from each column, and subjected to SDS-PAGE. rhTSG-6 wild type (FIG. 5B) and rhTSG-6-LINK (FIG. 5C) were detected in the fractions. Multiple bands are detected with TSG-6-LINK (FIG. 5C) because of varying degrees of glycosylation.

The peak fractions from the last column either were frozen directly at −80° C. for storage or buffer exchanged by dialysis with 200 mM NaCl/50 mM Tris-HCl buffer before freezing.

Bioassay of Recombinant Proteins in Chemically Injured Corneas

The experimental protocols were approved by the Institutional Animal Care and Use Committee of Texas A&M Health Science Center. Six-week-old male Lewis rats (LEW/Crl; Charles River Laboratories International, Inc.) weighing 180-200 g were used in all experiments. Rats were anesthetized by isoflurane inhalation. To create the chemical burn, 100% ethanol was applied to the whole cornea including the limbus for 15 seconds followed by rinsing with 10 ml of balanced salt solution. Then, the whole corneal and limbal epithelium was mechanically scraped using a surgical blade. Upon completion of the procedure, the eyelids of a rat were closed with one 8-0 silk suture at the lateral one third of the lid margin. At predetermined time points after injury, five rats each received injections of rh TSG-6 or rhTSG-6-LINK, each of which has a “His-tag” of six amino acid residues at the C-terminus (350 ng in 54 of PBS) obtained as hereinabove described, or the same volume of PBS was injected into the anterior chamber of the eyes of five rats. All injections were done with 32 gauge needle and syringe. Five uninjured (normal) rats served as controls.

After injury and treatment, the rat corneas were examined for corneal opacity and neovascularization under a dissecting microscope and photographed. Corneal opacity was assessed and graded by a blinded investigator who was an ophthalmologist as: grade 0, completely transparent cornea; grade 1, minimal corneal opacity, but iris clearly visible; grade 2, moderate corneal opacity, iris vessels still visible; grade 3, moderate corneal opacity, pupil margin but not iris vessels visible; and grade 4, complete corneal opacity, pupil not visible. For semi-quantitative estimate of neutrophil infiltration by assay for myeloperoxidase activity (MPO), the cornea was sectioned into small pieces and lysed in 150 μl of tissue extraction reagent containing protease inhibitors (Invitrogen). The supernatant was assayed for levels of pro-inflammatory cytokines and chemokines with commercial ELISA kits for IL-1β (Quantikine Kit; R & D Systems), and for MPO. (Rat MPO ELISA kit; HyCult biotech).

As shown in FIG. 6A, corneal opacity was reduced significantly in both rhTSG-6 and rhTSG-6-LINK-treated corneas. For an estimate of neutrophil infiltration, the concentration of myeloperoxidase (MPO) was assayed. Treatment with rhTSG-6 or rhTSG-6-LINK reduced the levels of MPO in the cornea significantly. (FIG. 6B). Also, the levels of the pro-inflammatory cytokine IL-1β were decreased significantly in the rhTSG-6 or rhTSG-6-LINK treated corneas as assayed by ELISA. (FIG. 6C).

The above results show that the rhTSG-6 and rhTSG-6-LINK proteins produced in accordance with the method of the present invention are effective in treating corneal injuries.

The disclosures of all patents, publications (including published patent applications), depository accession numbers, and database accession numbers are incorporated herein by reference to the same extent as if each patent, publication, depository accession number, and database accession number were specifically and individually incorporated by reference.

It is to be understood, however,that the scope of the present invention is not to he limited to the specific embodiments described above. The invention may be practiced other than as particularly described and still be within the scope of the accompanying claims.

What is claimed is:

1. A method of producing a biologically active protein or polypeptide, or biologically active fragment, derivative or analogue thereof, comprising:
(a) introducing into mammalian cells a polynucleotide encoding a biologically active protein or polypeptide or a biologically active fragment, derivative, or analogue thereof;
(b) culturing said cells by suspending said cells in a protein-free medium, wherein said medium includes at least one agent that suppresses production of hyaluronic acid or hyaluronan or a salt thereof by said cells, wherein said cells are cultured for a time sufficient to express said biologically active protein or polypeptide, or a biologically active fragment, derivative, or analogue thereof; and
(c) recovering said expressed biologically active protein or polypeptide, or a biologically active fragment, derivative, or analogue thereof from said cells.

2. The method of claim 1 wherein said mammalian cells are CHO cells.

3. The method of claim 1 wherein said biologically active protein or polypeptide is TSG-6 protein or a biologically active fragment, derivative, or analogue thereof.

4. The method of claim 3 wherein said TSG-6 protein or biologically active fragment, derivative, or analogue thereof has at least one histidine residue at the C-terminal thereof.

5. The method of claim 4 wherein said TSG-6 protein or biologically active fragment, derivative, or analogue thereof has 6 histidine residues at the C-terminal thereof.

6. The method of claim 1 wherein said at least one agent that suppresses production of hyaluronic acid or hyaluronan or a salt thereof by said cells is 4-methylumbelliferone.

7. A biologically active protein or polypeptide, or biologically active fragment, derivative, or analogue thereof produced by the method of claim 1.

8. A composition comprising:
(a) the biologically active protein or polypeptide, or biologically active fragment, derivative, or analogue thereof of claim 7; and
(b) an acceptable pharmaceutical carrier.

9. A method of producing a biologically active protein or polypeptide, or biologically active fragment, derivative, or analogue thereof, comprising:
(a) introducing into mammalian cells a polynucleotide encoding a biologically active protein or polypeptide, or a biologically active fragment, derivative, analogue thereof;
(b) culturing said cells by suspending said cells in a medium which includes at least one agent that suppresses production of hyaluronic acid or hyaluronan or a salt thereof by said cells, wherein said cells are cultured for a time sufficient to express said biologically active protein or polypeptide, or a biologically active fragment, derivative, or analogue thereof; and
(c) recovering said expressed biologically active protein or polypeptide, or a biologically active fragment, derivative, or analogue thereof, from said cells.
